(19) United States
(12) Patent Application Publication
(10) Pub. No.: US 2005/0153383 A1
(43) Pub. Date: Jul. 14, 2005

(54) Synthetic and recombinant substrates for the detecion of the von willebrand factor-cleaving protease

(76) Inventors: Robert R. Montgomery, Cedarburg, WI (US); Thomas J. Raife, Iowa City, IA (US); Sandra L. Haberichter, Greenfield, WI (US)

Correspondence Address: QUARLES & BRADY LLP, 411 E. WISCONSIN AVENUE, SUITE 2040, MILWAUKEE, WI, 53202-4497, US

(21) Appl. No.: 10/333,506

(22) PCT Filed: Jul. 27, 2001
(86) PCT No.: PCT/US01/23784

Related U.S. Application Data
(60) Provisional application No. 60/221,566, filed on Jul. 28, 2000.

(51) Int. Cl. C12Q 1/37

(52) U.S. Cl. 435/23

(57) ABSTRACT

A method of detecting von Willebrand Factor-cleaving protease in a test sample is disclosed. In one embodiment, the method comprises the steps of (a) obtaining a test bodily fluid sample; (b) exposing the test sample to a monomeric von Willebrand Factor fragment, wherein the fragment comprises amino acids 842 and 843, wherein cleavage of the fragment will occur proportional to the amount of protease in the sample; and (c) comparing the cleavage products to a standard curve and determining the amount of von Willebrand Factor-cleaving protease in the test sample.

### CROSS-REFERENCE TO RELATED APPLICATION

This application claims priority to U.S. provisional application Ser. No. 60/221,566, filed Jul. 28, 2000. Ser. No. 60/221,566 is incorporated by reference herein.

### STATEMENT REGARDING FEDERALLY SPONSORED RESEARCH OR DEVELOPMENT

### BACKGROUND OF THE INVENTION

A protease, activated in the presence of barium and other metal ions has been demonstrated to degrade full-length multimeric vWF into multimers of smaller size and into lower molecular weight peptides. This protease has been termed vWF-cleaving protease, hereinafter referred to as the “protease”. The activity of this protease has been demonstrated to be reduced in patients with Thrombotic Thrombocytopenia Purpura (TTP).

Current assays for the presence or absence of this protease utilize a cumbersome technique in which plasma from a patient is incubated with exogenous multimeric vWF in the presence of barium chloride on the surface of a membrane floating on a buffer containing 1.5 molar urea. Alternative strategies have recently been developed that utilize guanidine HCI in a tube assay or use substrate vWF that has been reacted with urea and then dialyzed to remove the urea so as to preserve the ability for the protease to cleave the vWF. One other modification has been reported that measures the loss of vWF multimers by collagen binding assays rather than multimeric analysis. In all instances, the readout of proteolysis is a loss of high molecular weight multimers and the production of cleavage fragments as an end-product of complete digestion. These terminal fragments are a C-terminal dimeric fragment of 350 kd and an N-terminal fragment of 200 kd. If multimers are incompletely digested, only one or the other fragment will in fact be separated from the rest of the multimeric chain up until the point of complete cleavage. If vWF is cleaved in half or a third, etc, no terminal fragments are produced. A terminal fragment is only produced when the terminal dimmer (C- or N-terminus) is cleaved. Thus, the fragment is not quanitative of protease activity.

### BRIEF SUMMARY OF THE INVENTION

The present invention began with our observation that if a vWF monomer were cleaved as an assay, the fragments would be produced quantitatively. The present invention will significantly increase the sensitivity and specificity of the protease assay that helps to establish the diagnosis of TTP and offers significant advances over current technology. The invention will greatly facilitate the turn-around time necessary for a result and will be used to identify the protease during purification processes.

In one embodiment, the present invention is a method of detecting von Willebrand Factor-cleaving protease in a test sample comprising the steps of (a) obtaining a test bodily fluid sample; (b) exposing the test sample to a monomeric von Willebrand Factor fragment, wherein the fragment comprises amino acids 842 and 843, wherein cleavage of the fragment will occur proportional to the amount of protease in the sample; and (c) comparing the cleavage products to a standard curve and determining the amount of von Willebrand Factor-cleaving protease in the test sample. Preferably the test bodily fluid sample is plasma and the monomeric von Willebrand Factor fragment is a recombinant molecule comprising between 10 amino acids and 100 amino acids in length.

In a preferred form of the present invention, the fragment is attached to a solid support, such as a microtiter dish or bead.

Other objects, advantages and features of the present invention will become apparent to one of skill in the after review of the specification, claims and drawings.

### BRIEF DESCRIPTION OF THE SEVERAL VIEWS OF THE DRAWINGS

FIG. 1 illustrates success at rendering full-length multimeric vWF susceptible to the protease without denaturation.

FIG. 2 demonstrates cleavage of a recombinant, truncated vWF molecule.

FIG. 3 demonstrates that mutations at the protease cleavage site block protease digestion of full-length vWF and that the insertion of the C4789T (nucleic acid nomenclature) caused increased digestion of full-length vWF.

FIG. 4 demonstrates the cleavage of FITC-labeled synthetic peptide.

FIG. 5 is a diagram of various vWF constructs.

FIG. 6 demonstrates multimeric and monomeric vWF and indicates points of clinical abnormalities of vWF as well as site-directed mutations that form alteration in vWF structure and function.

### DETAILED DESCRIPTION OF THE INVENTION

We envision that the proteolytic activity of the protease could be more precisely and quickly identified using one of several alternatives. The first alternative is to make use of three classes of mutations in the vWF cDNA that change vWF into a single polypeptide chain that we will refer to as a vWF monomer. In addition, mutations can be inserted into this monomer so as to increase the susceptibility of this monomer to this specific protease. Specific mutations are summarized below in Table 1.

TABLE 1

| | | new amino | |
|---|---|---|---|
| | | acid | |
| | | designation | old |
| | nucleic acid | numbering | designation |
| prevents n-terminal | A260C | Yp87S | |
| multimerization | | | |
| prevents c-terminal | T8416C | C2043R | |
| dimerzation | | | |
| prevents cleavage | TAC4813- | Y1605A | Y842A |
| | 4815GCA** | | |
| prevents cleavage | A4814T | Y1605F | Y842F |
| enhances cleavage | C4789T | R1597W | R834W |
| enhances cleavage | C4517T | S1506L | — |
| enhances cleavage | G4735A | G1579R | — |

**because of wobble other mutations could include GCU.

Second, we can produce synthetic truncated vWF monomers that contain an epitope tag on either the N or the C-terminus, or both. This permits the specific identification of the cleavage products.

Third, synthetic peptides containing the protease site can be developed by synthetic means and derived so as to place an epitope tag on either the C-terminus or the N-terminus that would produce a colorimetric determination of peptide cleavage. In all cases, proteolysis would be undertaken in the presence of inhibitors of other proteases that might confound the result for the specific protease of interest.

Various numbering systems have been developed for identifying the DNA directing the synthesis of vWF and the amino acid numbering to describe the protein sequence. For the purposes of this application, the cDNA will be numbered from beginning with the initiator methionine of the signal sequence that is encoded by the ATG at positions 1, 2, and 3. The full-length pre-pro-vWF cDNA is 8,439 nucleic acids in length. The protein sequence is numbered most commonly using amino acid 1 to represent the N-terminal serine of the mature vWF molecule. Thus, the mature vWF molecule spans amino acids 1 through amino acid 2,050. The propolypeptide of vWF is numbered from the initiator, methionine, preceded by a “p”. Thus, the initiator methionine is the p1 position and the C-terminus of the propeptide is arginine p763.

General Description of a Preferred Fragment

A preferred monomeric fragment of vWF starts at amino acid position one, at the beginning of the D′ domain, and concludes at amino acid position 1109, at the end of the A3 domain. The fragment is preferably expressed trans to avoid n-terminal multimerization. This fragment or a similar fragment includes a tyrosine at amino acid position 842. Cleavage of the fragment by the vWF multimerase or multmerase-containing plasma or other bodily fluids produces two digestion productions separated at position 842 and 843.

We envision that synthetic peptides of variable lengths may have increased sensitivity to specific protease. Such peptides could vary between 10 amino acids in length and 100 amino acids in length including the sequence surrounding the 842-843-cleavage site.

General Description of the Preferred Assay

A plasma or other bodily fluid sample (such as serum or saliva) being examined for its multimerase activity is first inhibited with a cocktail of protease inhibitors, then subsequently activated with a divalent cation. A preferred commercial protease inhibitor cocktail is Pefabloc (Boerhinger Mannheim). A preferred divalent cation is barium zinc or other heavy metals.

The fragment described above is then mixed with the activated plasma or other bodily fluid and deposited on a VSWP filter floating on a bath of a urea-based buffer. The VSWP filter is commercially made by Millipore. The urea is typically a 1.5 M solution.

After a 15-18 hour incubation at 37° C., the assay is stopped by the addition of EDTA (typically 0.2 M). One might use other unfolding agents, such as ristocetin or botrocetin. The sample is then boiled, electrophoretically separated in an acrylamide gel and then transferred to and immobilized on a membrane. A monoclonal antibody that binds to the N-terminus portion of the fragment and, if present, the N-terminus digestion product is allowed to bind to the immobilized polypeptides. One preferred n-terminus-specific antibody (MBC 105.4) is available from The Blood Center of Southeastern Wisconsin (Milwaukee, Wis.). Other commercially available antibodies may work (Dako P266) that are already conjugated. This specific monoclonal is then detected by an enzyme-conjugated antibody that binds to the monoclonal and is able to convert a substrate into a chemiluminescent product.

The assay may include a solid support, such as an ELISA tray or beads, to which is coupled, directly or indirectly, the N-terminus of a synthetic peptide to which an epitope or fluorochrome was covalently attached to the C-terminus. Alternatively, the C-terminus of the peptide could be coupled, directly or indirectly, to the support and the fluorochrome or epitope placed on the N-terminus.

If an ELISA tray were used, serial dilutions of the patient sample would be made to the wells of the modified ELISA tray. Barium chloride would then be added to the wells to activate the protease and after a set length of time the material would be removed and the wells washed with an appropriate buffer. An enzyme conjugated to a ligand that binds the epitope or fluorochrome would then be added. Again, after a set length of time the material would be removed and the wells washed with an appropriate buffer.

A substrate converted to a colored product by the enzyme conjugate is then added.

After a set length of time the reaction would be stopped and absorbence values are measured. The amount of color would be inversely proportional to the amount of cleavage that took place. As a negative control, a similar peptide containing mutations at either to both of positions 842 and/or 843 could be used since these alterations would preclude peptide cleavage and would demonstrate cleavage of the vWF peptide at some position other than the specific protease site. We would envision that such an assay could be performed in several hours rather than the current several days required for the assay.

Amino acid substitutions that replace the tyrosine in position 842 of the vWF fragment make the fragment resistant to cleavage by the vWF multimerase-containing plasma as tested in the current assay. In contrast, the amino acid substitution of a tryptophan for an arginine in position 834 of the vWF fragment enhances cleavage by the vWF multimerase-containing plasma. Other amino acid substitutions may similarly inhibit or enhance cleavage of the vWF fragment or fragments like it.

The vWF fragment does not spontaneously degrade, but is digested in a dose-dependent manner when the multimerase source is titrated into the current assay.

The vWF fragment is not digested in the current assay when the plasma source material does not contain a functional multimerase as may be found in clinical samples from patients with thrombotic thrombocytopenic purpura or similar disorders. The absence of multimerase function could be due to the absence of the multimerase, a present but dysfunctional multimerase or a function multimerase that is inhibited by an antibody or other inhibitory substance that interferes with reactivity.

The use of the standard curve gives the measure of quantitation to the assay.

### EXAMPLES

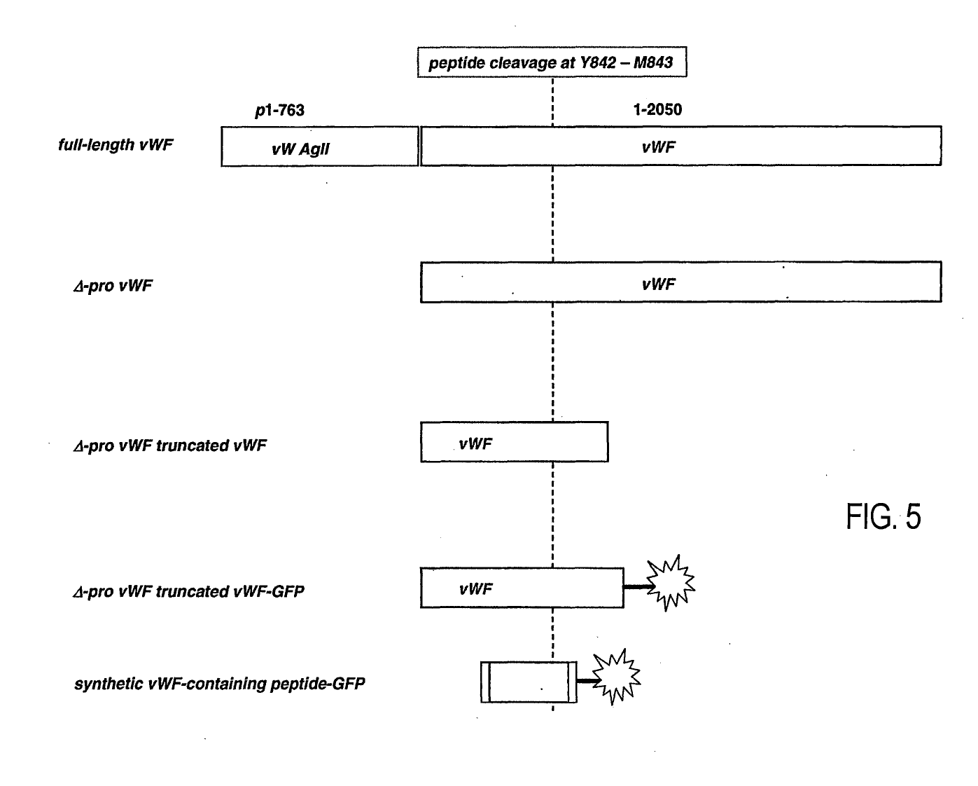

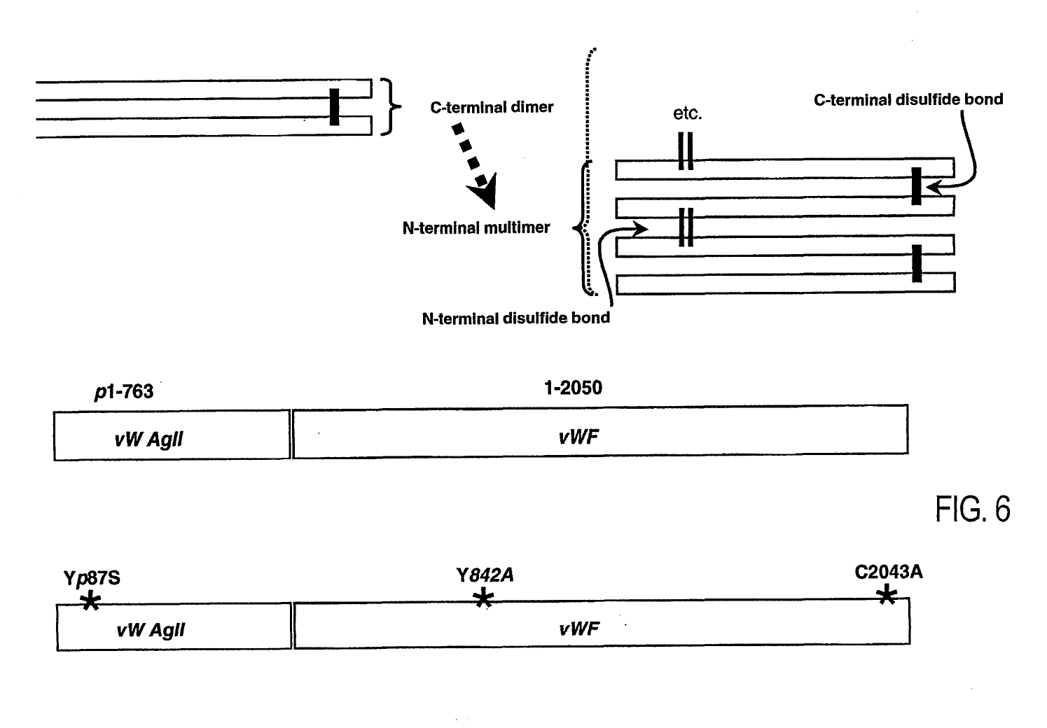

The laboratory of Robert Montgomery and colleagues was one of the laboratories to initially sequence vWF and to identify vW AgII as the propolypeptide of vWF. We have undertaken numerous studies to identify mutations in the vWF gene that cause clinical abnormalities of vWF as well as to produce site-directed mutations that confer alteration in vWF structure and function (see FIGS. 5 and 6). Specifically, we found that a mutation of the propolypeptide, Yρ87S mutation causes a loss of N-terminal multimerization of mature vWF. Only C-terminal vWF dimers and not higher molecular weight multimers are produced. In other experiments, we demonstrated that a mutation of the C-terminus of mature vWF, C2043R produces a vWF that does not C-terminal dimerize. Placing these two mutations together into a full-length vWF expression vector produces vWF that is monomeric (see FIGS. 5 and 6). We have demonstrated that this monomeric vWF still requires mild denaturation in order to make the vWF monomers susceptible to protease digestion.

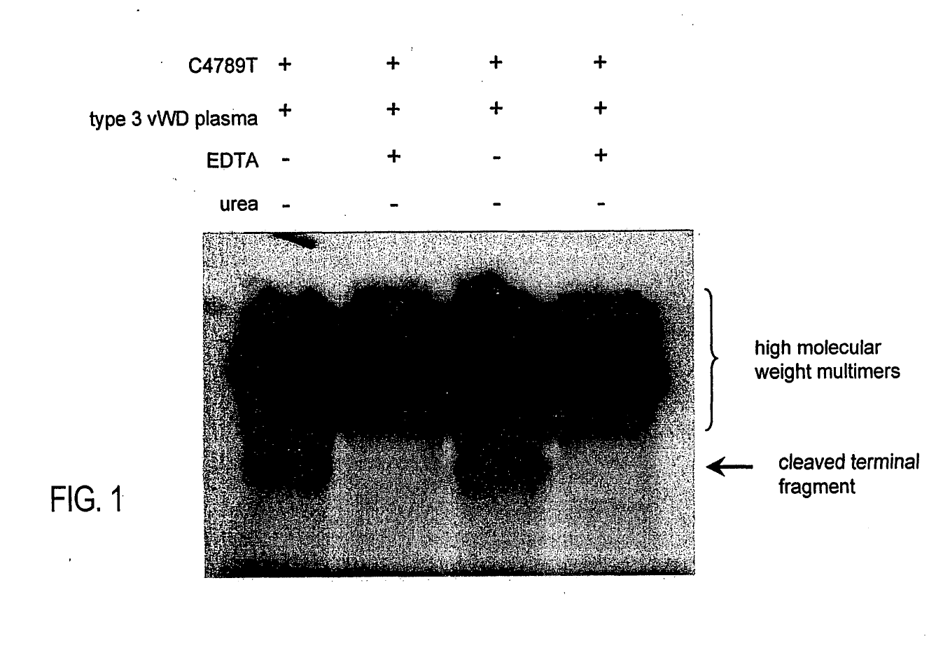

To further develop a molecule that will not require denaturation, we have inserted a mutation C4789T that changes an arginine to a tryptophan, which increases the protease susceptibility of full-length vWF. FIG. 1 illustrates success at rendering full-length multimeric vWF susceptible to the protease without denaturation.

As described above, we will insert this mutation into the monomeric form of vWF and expect to produce a monomeric vWF molecule that does not require significant denaturation to facilitate assay of the protease.

Since methods of detection still require assaying the cleavage fragments by either polyacrylamide gel, electrophoresis and Western blotting, we will place an epitope tag such as C-Myc, and a hemagglutinin (HA) tag, a flag-epitope, or green fluorescent protein (GFP) on either the C or the N-terminus. We have successfully made full-length vWF with these epitope tags on the C-terminus. As embodied in an assay, the N-terminus of monomeric vWF will be bound directly to a bead or indirectly through an monoclonal antibody with specificity for the N-terminus of vWF. Since this is monomeric vWF, cleavage at the protease site between position Y842 and M843 will result in the solubilization of the C-terminal fragment containing the C-terminal epitope that may then be directly quantitated in solution using either an enzyme-linked monoclonal antibody to the epitope or using an FITC or GFP detection system to assay the amount of cleavage without further handling except for the aspiration of the supernate. Alternatively, the beads containing the full-length monomeric vWF with the C-terminal epitope tag would be centrifuged and the amount of color remaining in the supernatant would be directly proportional to the peptide cleavage.

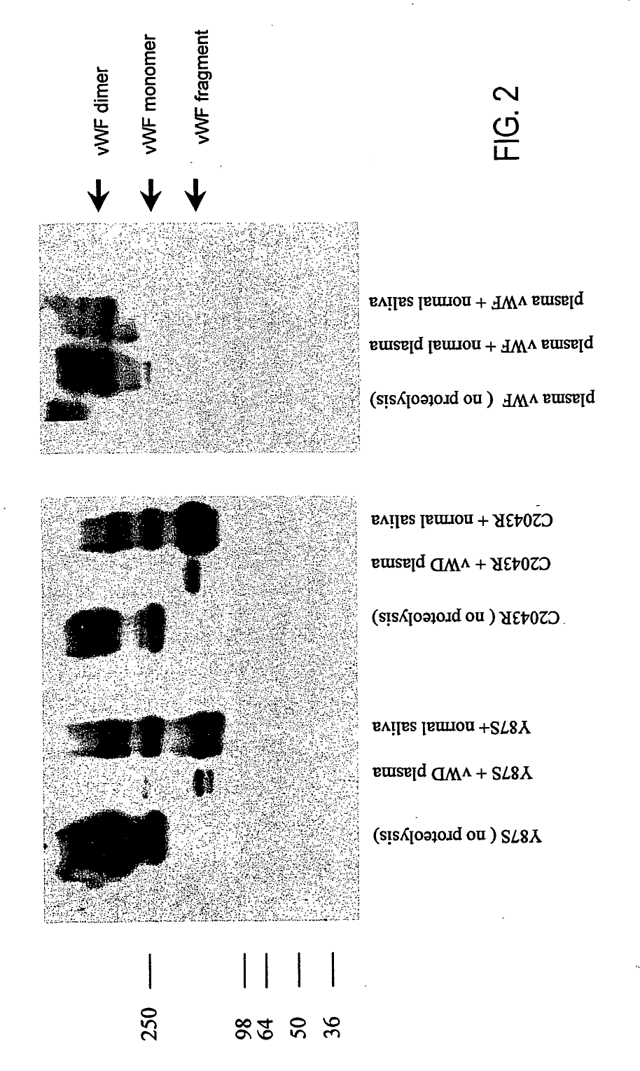

A second method makes use of truncated vWF molecules that we have produced that make vWF monomers that are truncated beyond the protease cleavage site at 842-843. Currently, we have a Δ-pro cDNA for vWF that includes the Y842A mutation together with the R834W mutation truncated beyond F1112 of mature vWF. This molecule therefore does not multimerize (absent propolypeptide), has increased susceptibility to protease (R834W), and has a green fluorescent protein on the C-terminal end (after F1112). Cleavage at the protease site produces an untagged polypeptide of 842 amino acids and a cleaved peptide of 270 amino acids containing the GFP tag on its C-terminus. FIG. 2 demonstrates cleavage of this recombinant truncated molecule.

In FIG. 2 we produced monomeric vWF using either the Yp87S or the C2043R mutations. These were susceptible to protease digestion. These have a GFP tag on the C-terminus but the western blot below uses an antibody stain for the GFP. On the right is plasma vWF stained with anti-vWF antibody showing absence of cleavage.

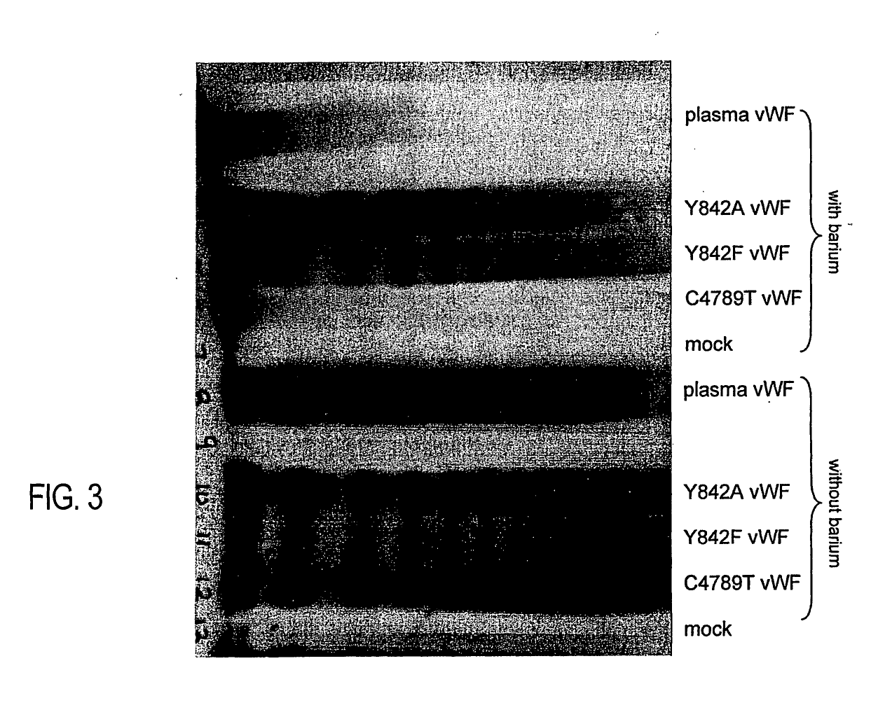

In both the first iteration and the second iteration, particular attention must be paid to non-specific cleavage of vWF. In order to assure specificity we have developed mutations at position 842 in which Y842 is changed to either an alanine or phenylalanine producing a molecule that is not cleaved by the metalloprotease seen in plasma. We envision using such constructs to rule out cleavage of vWF at a site other than the specific 842-843 cleavage. (See FIG. 3)

Referring to FIG. 3, recombinant vWF was mixed with type 3 vWD plasma as a source of protease in the presence and absence of barium. Mutating the vWF at position 842 disrupts the protease site rendering the vWF uncleavable at this site. This vWF can be used as a control for cleavage of vWF at a site other than 842-843. Note also that the C4789T mutation produces vWF with increased susceptability to cleavage.

The third alternative strategy to detect the protease utilizes synthetic peptides that are constructed so as to include the protease site at positions Y842-M843. We developed a synthetic peptide that was synthesized with the following sequence:

AGGGGLRYLSDHSFLVSQGDRGQAPNLVYMVYGNTASLA

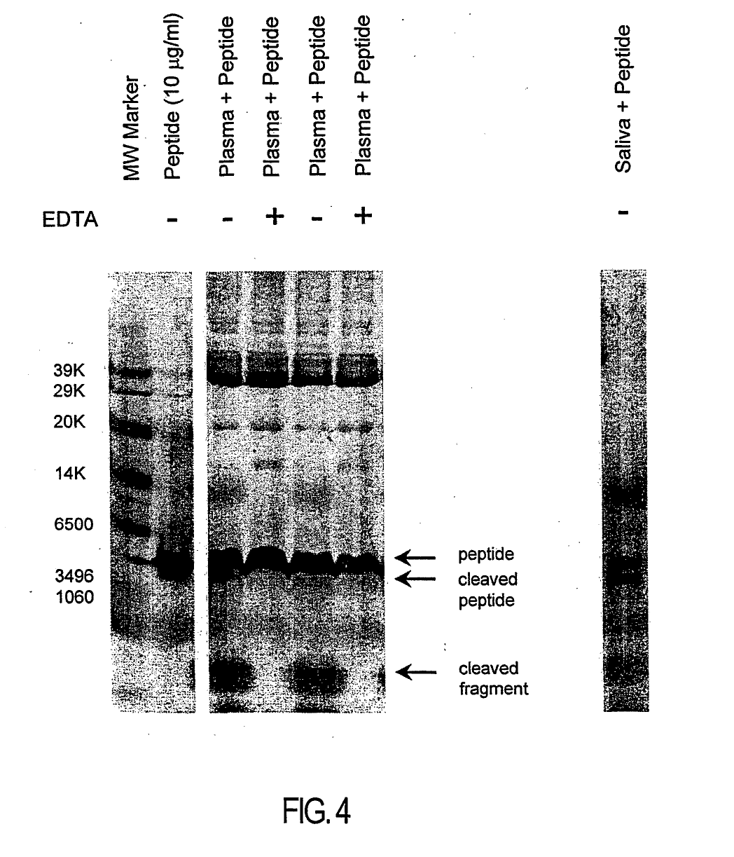

In this peptide, the sequence 1 through 5 was added so that an FITC could be added to the N-terminal alanine. The lucine at position 6 through the serine at position 37 correspond to the mature vWF sequence of L819-S850. The terminal L38 and A39 were added to facilitate FITC labeling of the C-terminus. FIG. 4 demonstrates cleavage of this peptide that is protease and divalent cation dependent. This synthetic peptide does not require denaturation but may be facilitated by incorporation of the R834W mutation into the sequence to further facilitate protease digestion. Although FIG. 4 demonstrates FITC labeled synthetic peptide, we would envision preferably labeling only one end and immobilizing the N-terminus onto a non-porous bead that could be then utilized as a substrate-containing reagent to assay protease activity. There is a direct relationship between the amount of labeled fragment released and the amount cleaved. Such a relationship is not present when multimeric vWF is used.

1. A method of detecting von Willebrand Factor-cleaving protease in a test sample, comprising the steps of
(a) obtaining a test bodily fluid sample;
(b) exposing the test sample to a monomeric von Willebrand Factor fragment, wherein the fragment comprises amino acids 842 and 843, wherein cleavage of the fragment will occur in a manner proportional to the amount of protease in the sample; and
(c) comparing the cleavage products to a standard curve and determining the amount of von Willebrand Factor-cleaving protease in the test sample.

2. The method of claim 1 wherein the bodily fluid sample is plasma.

3. The method of claim 1 wherein the fragment comprises between 10 and 100 amino acids.

4. The method of claim 1 wherein the bodily fluid sample is treated with a protease cocktail to suppress extraneous proteases.

5. The method of claim 1 wherein the fragment comprises a mutation Yp87S.

6. The method of claim 1 wherein the fragment comprises mutation C2043R.

7. The method of claim 1 wherein the fragment comprises mutation Y842A.

8. The method of claim 1 wherein the fragment comprises mutation Y842F.

9. The method of claim 1 wherein the fragment comprises mutation C4789T.

10. The method of claim 1 wherein the test sample is activated with a divalent cation, wherein the cation is a heavy metal.

11. The method of claim 10 wherein the divalent cation is selected from the group consisting of barium and zinc.

12. The method of claim 1 wherein the product of step (b) is deposited on a filter.

13. The method of claim 12 wherein the filter is then floated in a bath of urea-containing buffer.

14. The method of claim 1 wherein the fragment is immobilized on a solid support.

15. The method of claim 14 wherein the solid support is a microtiter plate.

16. The method of claim 14 wherein the solid support is a bead.

17. The method of claim 1 wherein the fragment is the translation product of a DNA molecule.


SEQUENCE LISTING (1 sequence)

SEQ ID NO: 1 - protein, 39 aa, Artificial Sequence
  Description of Artificial Sequence  Synthetic Peptide
AGGGGLRYLSDHSFLVSQGDRGQAPNLVYMVYGNTASLA